(19) United States
(12) Patent Application Publication
(10) Pub. No.: US 2016/0264876 A1
(43) Pub. Date: Sep. 15, 2016

(54) GASOLINE PREPARED FROM BIOMASS-DERIVED LEVULINIC ACID

(71) Applicant: The Regents of the University of California, Oakland, CA (US)

(72) Inventors: Mark Mascal, Oakland, CA (US); Saikat Dutta, Lakshya, East Mindapore (IN); Inaki Gandarias, Bilbao (ES)

(21) Appl. No.: 15/153,729

(22) Filed: May 12, 2016

Related U.S. Application Data
(63) Continuation of application No. PCT/US14/65798, filed on Nov. 14, 2014.
(60) Provisional application No. 61/904,876, filed on Nov. 15, 2013.

(51) Int. Cl. C10G 3/00; C07D 307/58

(52) U.S. Cl. CPC C10G 3/47; C10G 3/50; C07D 307/58; C10G 2400/06

(57) ABSTRACT

The present invention provides methods for preparing a C₆-C₁₀ alkane, and mixture thereof. The methods include forming a reaction mixture containing an angelica lactone dimer, a catalyst, and a hydrogen source under conditions sufficient to reduce the angelica lactone dimer, thereby preparing the alkane. The methods can be used to prepare branched alkanes useful for fuels. Methods for preparing an angelica lactone, methods for preparing an angelica lactone dimer, and methods for reducing a lactone to an alkane are also described.

### CROSS-REFERENCES TO RELATED APPLICATIONS

The present application is a continuation of International Patent Application No. PCT/US14/65798, filed Nov. 14, 2014, which claims priority to U.S. Provisional Patent Application No. 61/904,876, filed Nov. 15, 2013; which applications are incorporated herein by reference in their entirety.

### BACKGROUND OF THE INVENTION

In the race to produce biomass-derived, hydrocarbon-based, drop-in automotive fuels, most effort has focused on the condensation chemistry of carbohydrate derivatives, which can provide molecules with extended carbon chain lengths (>C₆) for deoxygenation to alkanes with hydrogen and a catalyst. One of the earliest efforts in this area was the aqueous phase reforming (APR) process, in which sugars and hydrogen reacted to give hydrocarbons, based on work originally described by Huber, Cortright, and Dumesic in 2004 and 2005 [Alonso et al., Chem. Soc. Rev. 2012, 41, 8075; Huber et al., Angew. Chem. Int. Ed. 2004, 43, 1549; Huber et al., Science 2005, 308, 1446]. Since then, related approaches have been reported by other groups, which have been summarized in recent reviews [Tompsett et al., Thermochemical Processing of Biomass 2011; 232, Serrano-Ruiz et al., Energy & Environmental Science 2011, 4, 83; Alonso et al., Green Chem. 2010, 12, 1493; Serrano-Ruiz et al., Ann. Rev. Chem. Biomol. Eng. 2010, 1, 79]. In many cases, the electrophile is a furfural, either 5-(hydroxymethyl)furfural (HMF) or furfural itself. These processes have inherent drawbacks in the poor availability of HMF on an industrial scale, and the lower relative abundance of C₅ sugars in biomass compared to C₆. In other instances, polyols, biogenic ketones, γ-valerolactone, or related molecules are the feedstock. In all cases, however, the products are linear alkanes or long-chain alkanes with single branches, and are generally described by the authors as renewable substitutes or additives for diesel or jet fuel. In no work that we are aware of, are branched alkanes suitable for drop-in use as gasoline produced by any of these methods.

Motor gasoline is a mixture of C₄ to C₁₂ n-alkanes and iso-alkanes along with cycloalkanes, arenes, and oxygenates. An important characteristic of gasoline is its antiknock index, which is estimated by measuring the octane rating of the fuel. Straight chain alkanes generally have octane numbers inferior to branched alkanes. For example, the Research Octane Number (RON) of hexane and its isomer 3-methyl pentane are 25 and 75, respectively [Motor Gasolines Technical Review, Chevron Corporation 2009]. Further branching gives even higher RONs. Regular unleaded gasoline is generally 87 octane in the US, with premium grades up to 93 octane. A higher octane rating allows for a higher compression ratio which translates to more power and better performance for the engine. Accordingly, methods for production of branched alkane fuels from renewable resources are needed. The present invention meets this and other needs.

### BRIEF SUMMARY OF THE INVENTION

In one aspect, the invention provides methods for preparing a C₆-C₁₀ alkane, or a mixture thereof. The methods include forming a reaction mixture containing an angelica lactone dimer, a catalyst, and a hydrogen source under conditions sufficient to reduce the angelica lactone dimer, thereby preparing the alkane.

In another aspect, the invention provides methods for preparing an angelica lactone dimer. The methods include forming a reaction mixture containing an angelica lactone and a catalytic amount of potassium carbonate under conditions sufficient to form the angelica lactone dimer, wherein the angelica lactone dimer is prepared in at least about 20% yield.

In another aspect, the invention provides methods for preparing an angelica lactone. The methods include forming a reaction mixture containing levulinic acid and a heterogeneous acid catalyst under conditions sufficient to lactonize the levulinic acid, thereby preparing the angelica lactone.

In another aspect, the invention provides methods for reducing a lactone to an alkane. The methods include forming a reaction mixture containing the lactone, a catalyst selected from Ir—ReOₓ/SiO₂ and Pt—ReOₓ/C, and hydrogen gas at a temperature of at least about 50° C. and a pressure greater than 1 bar, thereby reducing the lactone to the alkane.

### BRIEF DESCRIPTION OF THE DRAWINGS

FIG. 1 shows a general scheme for the synthesis of branched alkanes from levulinic acid according to the methods of the invention.

FIG. 2 shows the yield of C₁₀ hydrocarbons (black bars) and the yield of C_{<10} hydrocarbons (grey bars) as a function of the reaction temperature. Reaction conditions: 2.0 g of angelica lactone dimer (ALD) 3, 0.30 g of Ir—ReOₓ/SiO₂, 54 bar H₂. Reaction times: 3 h at 300° C., 6 h at 240° C., and 7h at 220 and 200° C.

FIG. 3A shows a GC-MS chromatogram of the liquid products obtained after hydrodeoxygenation of angelica lactone dimer 3. Conditions: 220° C., 7 h, 54 bar H₂, 2.0 g angelica lactone dimer, 0.3 g Ir—ReOₓ/SiO₂ catalyst.

FIG. 3B shows the GC-MS oven temperature profile for the analysis of the hydrocarbon products.

FIG. 4 shows the ¹H NMR spectrum of 3-ethyl-4-methylheptane in CDCl₃.

FIG. 5 shows the ¹³C NMR spectrum of 3-ethyl-4-methylheptane in CDCl₃.

### DETAILED DESCRIPTION OF THE INVENTION

### I. General

The present invention provides a high-yielding, three-step preparation of gasoline-like, branched C₆-C₁₀ hydrocarbons using biomass-derived levulinic acid as the sole starting material. The invention is based, in part, on the discovery that bimetallic catalysts (containing, for example, iridium metal and rhenium oxide) can promote the hydrodeoxygenation of lactones such as angelica lactone which, in turn, can be prepared from levulinic acid. Reaction conditions can be varied to control the distribution of alkane products obtained, and valuable synthetic intermediates can be prepared in high yield.

### II. Definitions

“C₆-C₁₀ Alkane” refers to a straight chain or branched chain, saturated, aliphatic hydrocarbon having from 6 to 10 carbon atoms. Examples of C₆-C₁₀ alkane include, but are not limited to, n-hexane, 3-ethyl-4-methylhexane, 3-methylhexane, n-heptane, 4-methylheptane, 3,4-dimethylheptane, 3-ethyl-4-methylheptane, n-octane, n-nonane, n-decane, and the like.

“Lactone” refers to a cyclic ester. The lactones can be prepared via intramolecular esterification of a hydroxyl-substituted carboxylic acid, although they can also be prepared by other methods. Lactones used in the methods of the present invention typically contain from about 5 to about 10 ring atoms (including the oxygen atom of the hydroxyl moiety of the corresponding carboxylic acid). The lactones typically contain from about 6 to about 10 carbon atoms, which can be saturated or unsaturated.

“Angelica lactone” refers to a-angelica lactone (i.e., 4-hydroxy-3-pentenoic acid γ-lactone, also known as 5-methyl-2(3R)-furanone; shown as formula A below), β-angelica lactone (i.e., 4-hydroxy-2-pentenoic acid γ-lactone, also known as 5-methyl-2(5H)-furanone; shown as formula B below), and mixtures thereof. The term “angelica lactone dimer” refers to the product of a reaction between two angelica lactone molecules. In some embodiments, the angelica lactone dimer is 5-methyl-5-(2-methyl-5-oxotetrahydrofuran-3-yl)furan-2(5H)-one.

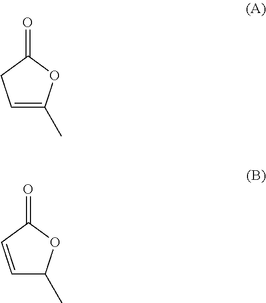

“Reducing a lactone” refers to conversion of a lactone (i.e., a cyclic ester) to one or more alkanes. In certain embodiments, reducing a lactone is conducted in the presence of a catalyst, under elevated temperature and/or pressure in the presence of H₂ gas. For example, angelica lactone dimer can be reduced to form C₆-C₁₀ alkanes in the presence of metal-metal oxide catalysts under conditions of high temperature and pressure in the presence of H₂ gas.

“Lactonization” refers to the formation of a lactone (i.e., cyclic ester) by intramolecular attack of a hydroxyl group on a carbonyl group. For example, levulinic acid undergoes intramolecular dehydration under acidic conditions to form angelica lactones. “lactonize” refers to the process of lactonization.

“Levulinic acid” refers to 4-oxopentanoic acid.

“Catalyst” refers to a substance that participates in a chemical reaction so as to increase the rate of the reaction, but which is itself not consumed in the reaction. Examples of catalysts include, but are not limited to, metals, metal oxides, metal complexes, acids, and bases. The catalysts used in the methods of the invention can be homogenous catalysts, which are present in the same phase as the other reaction components (such as, for example, in solution). The catalysts used in the methods of the invention can also be heterogenous catalysts. Heterogenous catalysts are typically present as solid materials (or immobilized on solid substrates) in reaction mixtures containing solution-phase reactants. “Heterogenous acid catalyst” refers to a solid material containing a plurality of catalytic Bronsted acid or Lewis acid moieties. Examples of heterogenous acid catalysts include, but are not limited to, sulfonic acid resins (e.g., Dowex-50) and montmorillonite clay. “Montmorillonite clay” refers to a monoclinic, smectite silicate clay having the general formula (Na, Ca)₀₃₃(Al, Mg)₂(Si₄O₁₀)(OH)₂.nH₂O.

“Forming a reaction mixture” refers to the process of bringing into contact at least two distinct species such that they mix together and can react, either modifying one of the initial reactants or forming a third distinct species, i.e., a product. It should be appreciated, however, that the resulting reaction product can be produced directly from a reaction between the added reagents or from an intermediate from one or more of the added reagents which can be produced in the reaction mixture.

“Hydrogen source” refers to a substance providing hydrogen atoms for transfer to a substrate molecule. Examples of hydrogen sources include, but are not limited to, hydrogen gas, metal hydrides, formic acid, and isopropanol.

“Inorganic base” refers to ammonia, ammonium salts, and basic compounds having a metal atom bound to one or more oxygen-, nitrogen-, or halogen-based groups. Examples of inorganic bases include, but are not limited to, calcium carbonate (CaCO₃), calcium hydroxide Ca(OH)₂, sodium bicarbonate (NaHCO₃), sodium carbonate (Na₂CO₃), sodium hydroxide (NaOH), potassium carbonate (K₂CO₃), and potassium hydroxide (KOH).

“About” and “around,” as used herein to modify a numerical value, indicate a close range surrounding that explicit value. If “X” were the value, “about X” or “around X” would indicate a value from 0.9X to 1.1X or a value from 0.95X to 1.05X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.98X.”

A. Methods for preparing C₆-C₁₀ Alkanes

In one aspect, the present invention provides a method for preparing a C₆-C₁₀ alkane, or a mixture thereof. The method includes forming a reaction mixture containing an angelica lactone dimer, a catalyst, and a hydrogen source under conditions sufficient to reduce the angelica lactone dimer, thereby preparing the alkane.

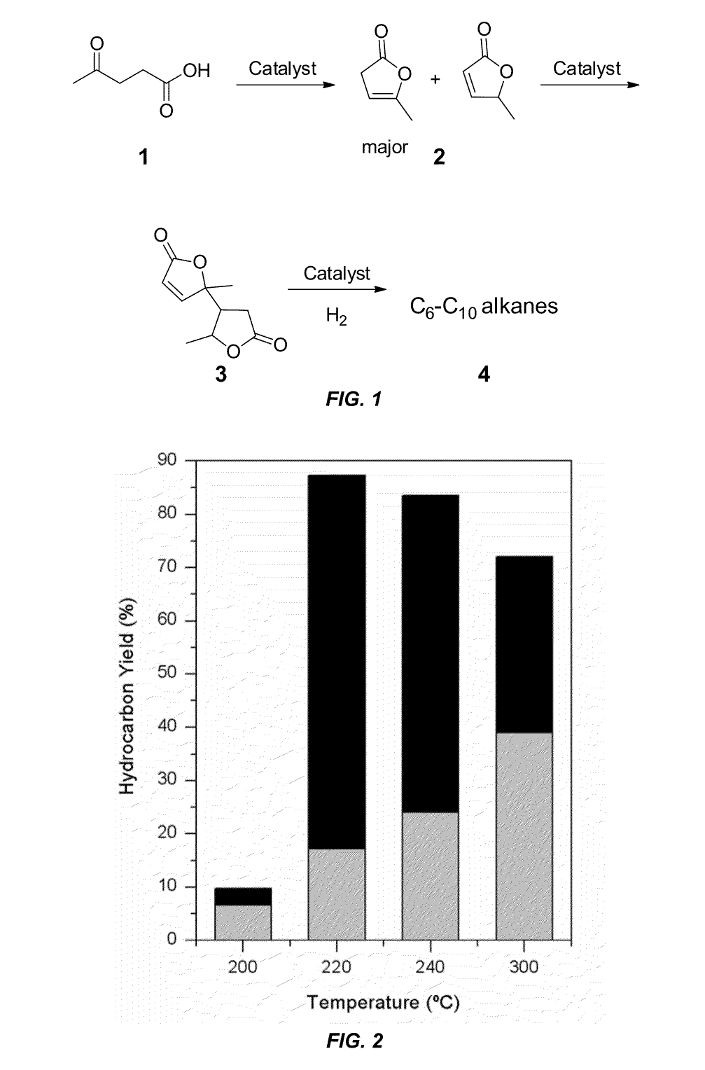

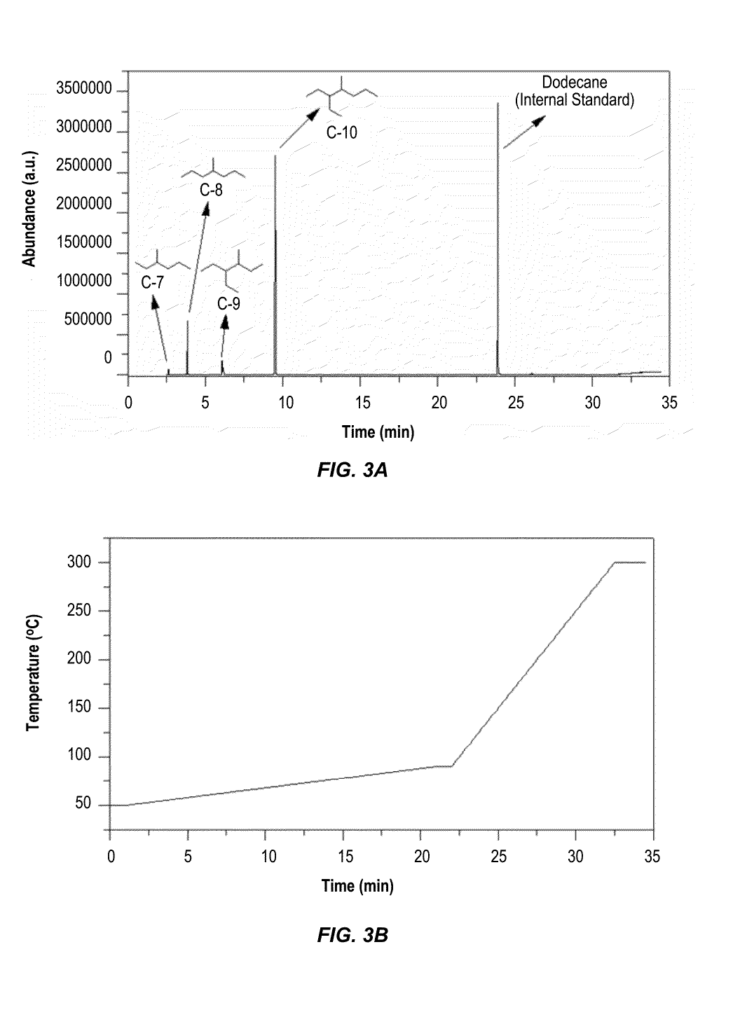

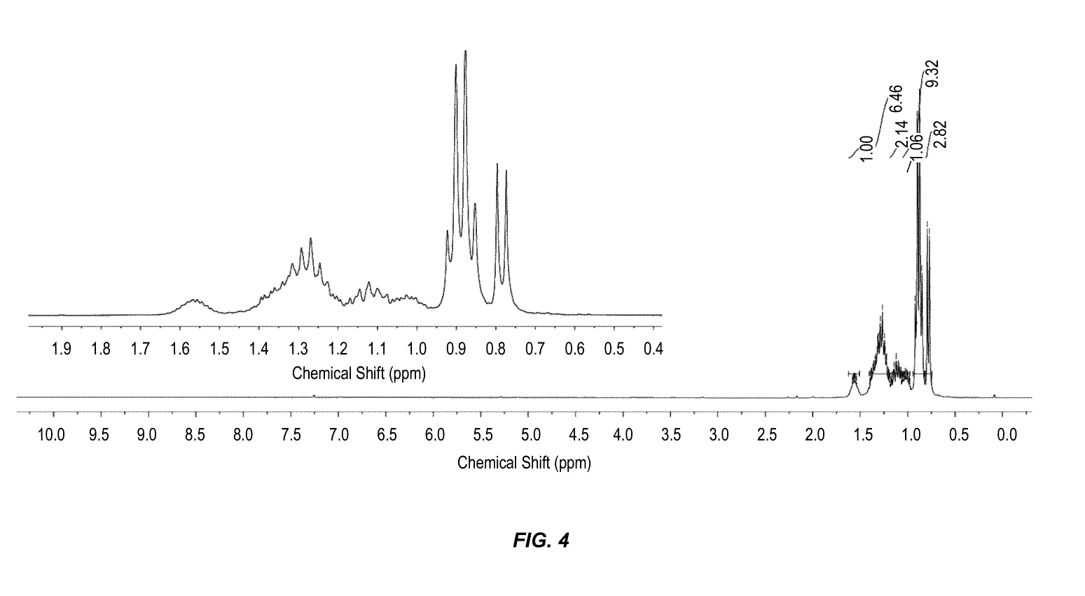

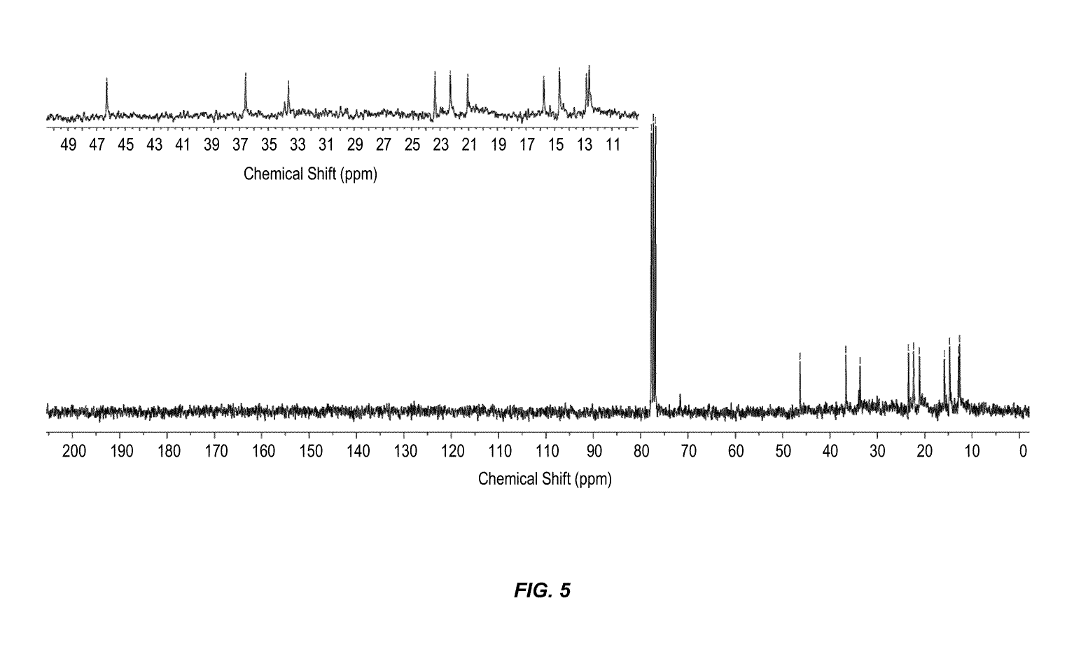

The angelica lactone dimer can be prepared from angelica lactone that is obtained from levulinic acid, a renewable resource. A general scheme for preparation of the alkane products from levulinic acid is outlined in FIG. 1. The reduction of angelica lactone 3 is summarized below in Scheme 1.

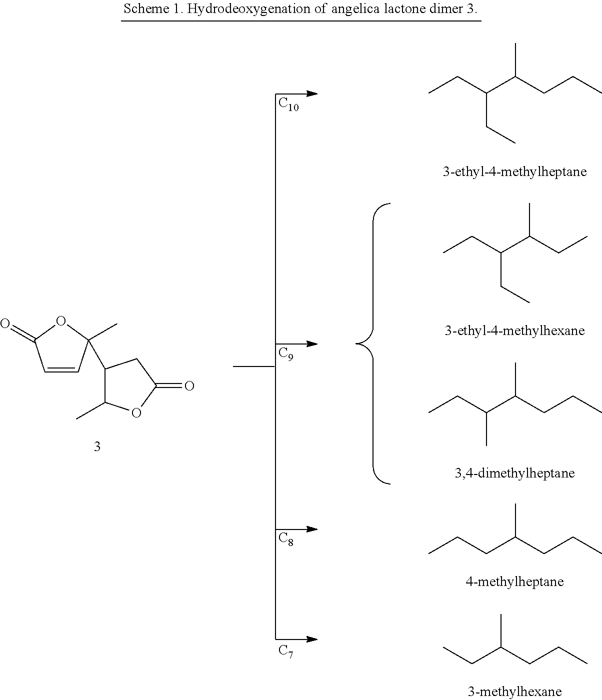

### Alkane Products

A number of alkane products can be prepared using the methods of the invention. The methods can be used to prepare various hexanes, heptanes, octanes, nonanes, and decanes, as well as mixtures thereof. The alkane can be a branched alkane, such as 3-ethyl-4-methylhexane, 3-ethyl-4-methylheptane, 3,4-dimethylheptane, 4-methylheptane, 3-methylhexane, and the like. Branched alkane products prepared according to the methods of the invention can be used as fuels with high octane ratings, which can provide increased power and superior performance for automotive engines and other machinery. In contrast, straight chain alkanes have octane numbers inferior to branched alkanes. For example, the Research Octane Number (RON) of linear n-hexane and its branched isomer 3-methylpentane are 25 and 75, respectively. Advantageously, the methods of the invention can be conducted using levulinic acid as starting material, which is a renewable resource derived from biomass.

In some embodiments, the C₆-C₁₀ alkane is selected from:

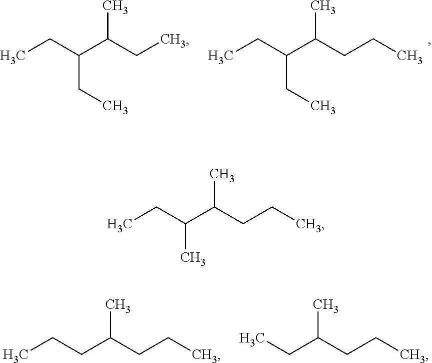

and mixtures thereof.

The methods of the invention provide alkane products in high yield. Complete conversion of angelica lactone dimer to alkane products is provided in certain embodiments, and C₆-C₁₀ alkanes are typically provided in yields of from about 10% to about 90%. In some embodiments, C₇-C₁₀ alkanes are provided in yields of from 10% to about 90%. The C₆-C₁₀ alkane yield or C₇-C₁₀ alkane yield can be for example, from about 15% to about 90%, or from about 40% to about 70%. Advantageously, the distribution of alkane products can be controlled by varying conditions such as temperature and hydrogenation pressure.

In some embodiments, the invention provides a method for preparing C₆-C₁₀ alkanes wherein the C₆-C₁₀ alkane includes at least about 40 mol C₁₀ alkanes. In some embodiments, the C₆-C₁₀ alkane includes at least about 60 mol C₁₀ alkanes. In some embodiments, the C₆-C₁₀ alkane includes at least about 80 mol C₁₀ alkanes. Depending on the particular reaction conditions and catalyst used, the methods can provide C₆-C₁₀ alkanes containing at least about 40, 45, 50, 55, 60, 65, 70, 75, 80, 85, 90, 95, or 99 mol % C₁₀ alkanes.

In general, angelica lactone dimers are used as precursor materials for the preparation of C₆-C₁₀ alkanes in the methods of the invention. The angelica lactone dimer can include any dimer formed from the reaction of two angelica lactone molecules, or a mixture of multiple dimers. In some embodiments, the angelica lactone dimer is selected from:

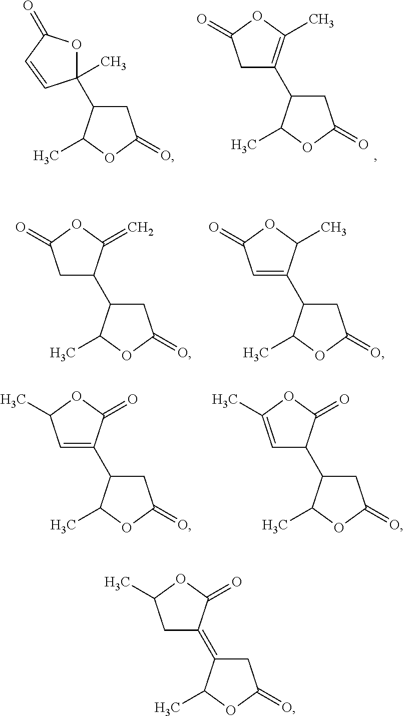

and mixtures thereof.

In some embodiments, the angelica lactone dimer is

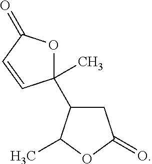

In some embodiments, the angelica lactone dimer consists essentially of

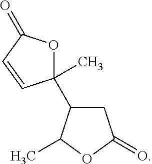

### Catalysts

A number of catalysts can be used for reducing the angelica lactone dimer. Metal-and metal oxide-based catalysts are particularly useful in the methods of the invention. Such catalysts include, but are not limited to, iridium-based catalysts, platinum-based catalysts, rhenium-based catalysts, copper-based catalysts, tungsten-based catalysts, and zinc-based catalysts. The catalyst can be a heterogenous catalyst, which is generally present on a solid support material that does not dissolve in the hydrogenation reaction mixture. Alternatively, the catalyst can be a homogenous catalyst, which is dissolved in the reaction mixture or which is otherwise in the same phase as the angelica lactone dimer.

Examples of homogenous catalysts include, but are not limited to: RuCl₂(PPh₃)₄; RuH₂(PPh₃)₄; RuH₂(CO)(PPh₃)₃; RuH(CO)Cl(PPh₃)₃; RuH(CF₃CO₂)(CO)(PPh₃)₂; RuCl₂(PPh₃)₄; RuCl₃; RhCl(PPh₃)₃; RhCl(COHPPh₃)₂; RhCl₃.3H₂O; RhH(PPh₃)₄; RhH(CO)(PPh₃)₃; IrHCl₂(Me₂SO)₃; IrHCl₂(CO)(PPh₃)₂; IrH₂Cl(PPh₃)₃; IrHCl₂(PPh₃)₃; IrH₃(PPh₃)₂; IrH₅(PPh₃)₃; IrCl(CO)(PPh₃)₂; IrBr(CO)(PPh₃)₂; IrI(CO)(PPh₃)₂; IrH(CO)(PPh₃)₃; IrH(COMPPh₃)₂; IrCl(C₈H₁₂)PPh₃; IrH[P(OPh)₃]₄; Os(CF₃CO₂)(CO)(PPh₃)₂; OsHCl(PPh₃)₃; OsH(CO)Cl(PPh₃)₃; PtCl₂(PPh₃)₂; PtCl₂/SnCl₂; K₂PtCl₄; PtCl₂(SnCl₂)(PPh₃)₂; cis-PtCl₂(PEt₃)₂; FeCl₂(PPh₃)₂; CoCl₂(PPh₃)₂; NiCl₂(Pn-Bu₃)₂; ReCl₅; and CoH[P(OPh)₃]₃. The homogenous catalyst can be a palladium catalyst such as a palladium(0) complex [e.g., tetrakis(triphenylphosphine)palladium(0)]; a palladium salt [e.g., palladium(II) acetate, palladium(II) chloride]; or a palladium(II) complex [e.g., allylpalladium(II) chloride dimer, (1,1′-bis(diphenylphosphino)ferrocene)-dichloropalladium(II), bis(acetato)bis(triphenylphosphine)palladium(II), bis(acetonitrile)dichloropalladium(II)].

Examples of heterogenous catalysts include, but are not limited to, pure bulk metals, finely divided metal powders, nanoparticles, porous particulate metals (also known as skeletal or sponge metals), Rieke metals, and metals dispersed on carriers such as carbon (e.g., activated charcoal) or inorganic salts (e.g., calcium carbonate, barium sulfate). Metal alloys containing two or more metals can also be used in bulk form, as powders, nanoparticles, and porous particles, or dispersed on carriers. Heterogenous catalysts include, but are not limited to: Ni (Raney), Pt/C, Pt (black), Rh/C, Rh (black), Ru (black), Ru/C, Ir (black), Pd/Ru, Ni/Cu, Os (black), Co (black), Fe (black), MgO/SiO₂, MgO, Al₂O₃, In, and Co/Mo/Al₂O₃.

In certain embodiments, the catalyst is a supported bimetallic catalyst containing a solid support and a combination of a metal and a metal oxide. Solid supports can include, for example, activated charcoal, zeolites, niobia, and silica or alumina particles. The solid support can be coated, impregnated, or otherwise associated with a mixture of a metal (such as iridium, platinum, and the like) and a metal oxide (such as rhenium oxide, tungsten oxide, and the like). In certain embodiments, the metal oxide is a low-valent metal oxide (i.e., an oxide wherein the metal is at an oxidation state of less than or equal to six but greater zero). Typically, the molar ratio of the metal to the metal oxide will range from about 0.01:1 to about 2:1.

A supported bimetallic catalyst can contain any suitable amount of metal. Typically, the supported bimetallic catalyst contains from about 0.1% to about 20% metal by weight. The supported bimetallic catalyst can contain, for example, from about 0.1% to about 10% metal by weight, or from about 10% to about 20% metal by weight, or from about 1% to about 15% metal by weight, or from about 2% to about 10% metal by weight, or from about 4% to about 8% metal by weight. The supported bimetallic catalyst can contain about 0.5%, 1%, 1.5%, 2%, 2.5%, 3%, 3.5%, 4, 4.5%, 5%, 5.5%, 6%, 6.5%, 7%, 7.5%, or about 8% metal by weight. In some embodiments, the supported bimetallic catalyst contains from about 0.1% to about 10% iridium by weight or from about 10% to about 20% iridium by weight. In some embodiments, the supported bimetallic catalyst contains from about 0.1% to about 10% platinum by weight or from about 10% to about 20% platinum by weight.

A supported bimetallic catalyst can contain any suitable amount of metal oxide. Typically, the supported bimetallic catalyst contains from about 0.1% to about 20% metal oxide by weight. The supported bimetallic catalyst can contain, for example, from about 0.1% to about 10% metal oxide by weight, or from about 10% to about 20% metal oxide by weight, or from about 1% to about 15% metal oxide by weight, or from about 2% to about 10% metal oxide by weight, or from about 4% to about 8% metal oxide by weight. The supported bimetallic catalyst can contain about 0.5%, 1%, 1.5%, 2%, 2.5%, 3%, 3.5%, 4, 4.5%, 5%, 5.5%, 6%, 6.5%, 7%, 7.5%, or about 8% metal oxide by weight. In some embodiments, the supported bimetallic catalyst contains from about 0.1% to about 10% rhenium oxide by weight or from about 10% to about 20% rhenium oxide by weight. In some embodiments, the supported bimetallic catalyst contains from about 0.1% to about 10% tungsten oxide by weight or from about 10% to about 20% tungsten oxide by weight. The metal oxides in the supported bimetallic catalysts typically have variable stoichiometry. For example, rhenium oxide designated “ReOₓ” can be present in a supported bimetallic catalyst as a mixture of ReO₂, ReO₃, and Re₂O₇. Similarly, tungsten oxide designated “WOₓ” can be present in a supported bimetallic catalyst as a mixture of W₂O₃, WO₂, and WO₃.

The composition of the supported bimetallic catalyst can be determined using a number of techniques known to those of skill in the art, including but not limited to X-ray diffraction analysis and X-ray absorption spectroscopy.

In some embodiments, the catalyst includes at least one member selected from CuO, ZnO, iridium metal, rhenium oxide (ReOₓ), platinum metal, tungsten oxide (WOₓ), and phosphotungstic acid. In some embodiments, the catalyst includes at least one member selected from mixed copper zinc oxide (Cu—ZnO); iridium-rhenium oxide (Ir—ReOₓ); platinum-rhenium oxide (Pt—ReOₓ); and platinum-tungsten oxide (Pt—WOₓ).

In some embodiments, the catalyst includes iridium-rhenium oxide on silica particles (Ir—ReOₓ/SiO₂). In some embodiments, the catalyst includes platinum-rhenium oxide (Pt—ReOₓ) on activated charcoal. In some embodiments, the catalyst includes mixed copper zinc oxide on alumina (Cu—ZnO/Al₂O₃).

Any suitable amount of catalyst can be used in the methods of the invention. Typically, a substoichiometric amount of catalyst with respect to the angelica lactone dimer is used in the hydrogenation reaction. That is, the number of moles of catalyst in the reaction mixture is less than the number of moles of starting material in the reaction mixture. The molar ratio of catalyst to starting material is generally less than 1:1. In some embodiments, the molar ratio of catalyst to starting material is less than 0.1:1. In some embodiments, the molar ratio of catalyst to starting material is less than 0.01:1. One of skill in the art will appreciate that the molar ratios set forth herein can also be expressed as mole % values and will know how to derive a mole % value from a molar ratio.

### Hydrogen Sources

Any suitable hydrogen source can be used in the methods of the invention. For example, hydrogen gas can be used as the hydrogen source. Hydrogen gas can be used as a pure gas, or as a mixture containing hydrogen gas and an inert gas such as argon or nitrogen. Other examples of hydrogen sources include, but are not limited to: hydrocarbons such as cyclohexene, cyclohexadiene, limonene, indane, and tetralin; alcohols such as ethanol, propan-2-ol, butan-2-ol, pentan-2-ol, benzyl alcohol, phenol, hydroquinone, diphenylmethanol, 1,2-ethanediol, 2,3-butanediol, and 1,2-cyclohexanediol; carboxylic acids such as lactic acid, ascorbic acid, mandelic acid, and formic acid, as well as salts of carboxylic acids such as triethylammonium formate; phosphorus oxoacids such phosphinic acid, and salts of phosphorus oxoacids such as sodium phosphinate; hydride reagents such as sodium borohydride; amines such as isopropylamine and isobutylamine; and other compounds such as hydrazine, hydroxylamine, dioxane, indoline, and N-benzylaniline.

In some embodiments, the hydrogen source is selected from hydrogen gas, a hydrocarbon, an alcohol, a carboxylic acid, a phosphorus oxoacid, and a hydride reagent. In some embodiments, the hydrogen source is selected from hydrogen gas, formic acid, and trimethylammonium formate. In some embodiments, the hydrogen source is hydrogen gas.

### Reaction Conditions

The hydrogenation reaction in the methods of the invention can be conducted at any suitable pressure. In general, hydrogenation reactions are conducted at pressures ranging between about 1 bar and about 75 bar. A hydrogenation reaction can be conducted, for example, at from about 1 bar to about 10 bar, or from about 10 bar to about 25 bar, or from about 25 bar to about 50 bar, or from about 50 bar to about 75 bar, or from about 10 bar to about 60 bar, or from about 20 bar to about 50 bar, or from about 30 bar to about 40 bar. A hydrogenation reaction can be conducted at about 1, 5, 10, 15, 20, 30, 35, 35, 40, 45, 50, 55, 60, 65, 70 or about 75 bar. One of skill in the art will appreciate that the reaction pressure use for a particular reaction will depend in part on the particular compound being hydrogenated, as well as on the characteristics and specifications of the equipment used for the hydrogenation reaction. In some embodiments, the reaction mixture is at a pressure of about 50 bar. In some embodiments, the reaction mixture is at a pressure of about 54 bar.

The hydrogenation reaction in the methods of the invention can be conducted at any suitable temperature. In general, hydrogenation reactions are conducted at temperatures ranging between about 20° C. and about 220° C. A hydrogenation reaction can be conducted, for example, at from about 20° C. to about 40° C., or from about 20° C. to about 100° C., or from about 40° C. to about 100° C., or from about 20° C. to about 150° C., or from about 100° C. to about 150° C., or from about 150° C. to about 220° C. A hydrogenation reaction can be conducted at about 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, or about 220° C. One of skill in the art will appreciate that reaction temperatures will depend in part on the particular compound being hydrogenated, as well as on the characteristics and specifications of the equipment used for the hydrogenation reaction. In some embodiments, the reaction mixture is heated to a temperature of from about 160° C. to about 210° C. In some embodiments, the reaction mixture is heated to a temperature of about 220° C.

### Conversion of Levulinic Acid to Alkane Products

As described in more detail below, some embodiments of the invention provide methods which further include forming a reaction mixture containing an inorganic base and an angelica lactone selected from:

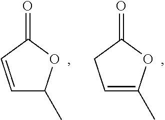

and mixtures thereof, under conditions sufficient to form the angelica lactone dimer.

In some embodiments, the inorganic base is K₂CO₃. Other inorganic bases, including but not limited to Cs₂CO₃, CaCO₃, Ca(OH)₂, Na₂CO₃, NaOH, and KOH, can also be used in the methods of the invention.

As described in more detail below, some embodiments of the invention provide methods which further include forming a reaction mixture containing levulinic acid and a heterogeneous acid catalyst under conditions sufficient to form the angelica lactone.

In some embodiments, the heterogeneous acid catalyst includes montmorillonite clay.

In some embodiments, the method of the present invention includes:
i) forming the reaction mixture containing levulinic acid and montmorillonite clay under conditions sufficient to form the angelica lactone selected from:

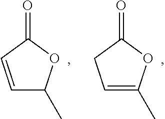

and mixtures thereof;
ii) forming the reaction mixture containing the angelica lactone and K₂CO₃ under conditions sufficient to form the angelica lactone dimer selected from

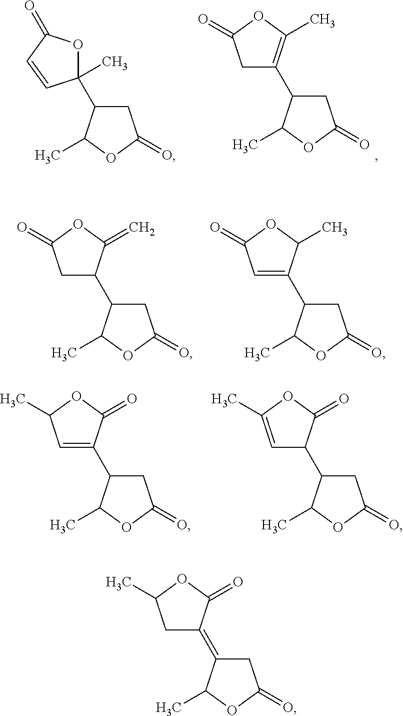

and mixtures thereof; and
iii) forming the reaction mixture containing the angelica lactone dimer, hydrogen gas, and a catalyst selected from Ir—ReOₓ/SiO₂ and Pt—ReOₓ/C, under conditions sufficient to form the C₆-C₁₀ alkane, wherein the C₆-C₁₀ alkane includes an alkane selected from:

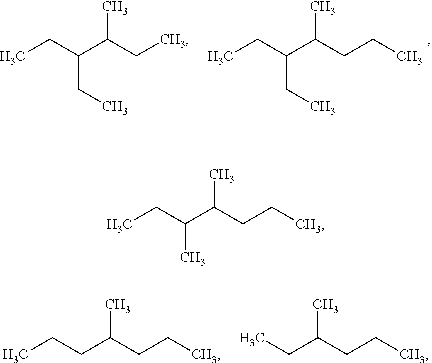

and mixtures thereof.

In a related aspect, the invention provides a C₆-C₁₀ alkane, or mixture thereof, which is prepared according to the methods above.

B. Methods for Preparing Angelica Lactone

In another aspect, the invention provides a method of preparing an angelica lactone. The method includes forming a reaction mixture containing levulinic acid and a heterogeneous acid catalyst under conditions sufficient to lactonize the levulinic acid, thereby preparing the angelica lactone, as shown in Scheme 2.

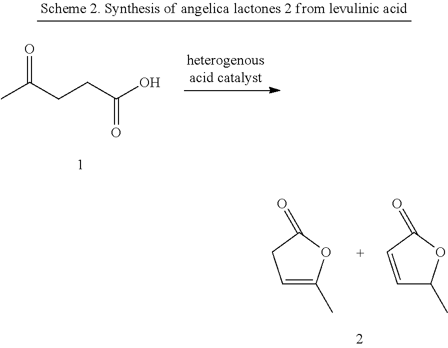

In some embodiments, the angelica lactone is selected from 5-methylfuran-2(5H)-one:

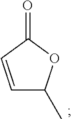

methylfuran-2(3H)-one:

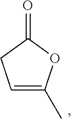

and mixtures thereof.

### Acid Catalysts

Any suitable acid can be used in the methods of the invention. Examples of useful acids include, but are not limited to, hydrochloric acid, sulfuric acid, nitric acid, acetic acid, trifluoroacetic acid, and sulfamic acid (also referred to as amidosulfonic acid and sulfamidic acid). The acid can also be a sulfonic acid such as methanesulfonic acid, trifluoromethanesulfonic acid, p-toluenesulfonic acid, and the like. Heterogenous acid catalysts can be particularly useful in the methods of the invention. A number of heterogenous acid catalysts can be used in the methods of the invention including, but not limited to: sulfated zirconia; tungstated zirconia; cation exchange resins (known to those of skill in the art by names including NKC-9, D002, and the like); gelular and microporous type ion-exchange resins (known to those of skill in the art by names including EBD 100, EBD 200, and the like); polyvinyl alcohol (PVA) cross-linked with sulfosuccinic acid and the like; heteropolyacids (e.g., H₃PW₁₂O₄₀, Cs_{2.5}H_{0.5}PW₁₂O₄₀, and the like); zeolites (e.g., H-ZSM5, mordenite zeolite, and the like); polyaniline sulfate on solid supports such as activated carbon; and sulfonic acid ion exchange resins (e.g., Dowex-50, Amberlyst-15, Amberlyst XN-1010, and the like).

In certain embodiments, the heterogenous acid is a mineral clay, such as montmorillonite, beidellite, nontronite, hectorite, saponite, sauconite, volkhonskoite, medmontite, pimelite, and the like. Montmorillonite-rich minerals, such as bentonite, can also be used in the methods of the invention.

The montmorillonites are crystalline clay minerals of the three-layer type and have an expanding lattice structure. These clays have a laminar or sheet structure wherein the repeating layers consist of two silica tetrahedra and a central alumina octahedron. The layers are continuous in one direction and stacked one above the other in the other direction. Montmorillonite clays have a micaceous structure with ultimate particle sizes typically less than 0.5 micron in maximum dimension. The laminar nature of the montmorillonite makes it possible for water and other polar molecules, including organic molecules, to enter between the layers causing the lattice to expand. Charge deficiencies often exist in the lattice of the montmorillonites as a result of substitution (exchange) between ions of unlike charge. The charge deficiencies within these clays can be balanced by the adsorption of cations (e.g. Na⁺, K⁺, Ca⁺⁺). Varying proportions of ions are found in its cation exchange positions, depending on the source of the material.

“Activated” clays can be used in the methods of the invention. Clay can be activated by the direct treatment of bentonite clays with mineral acids at elevated temperatures. For example, use of a 15% sulfuric acid solution can be used. The acid-treated materials can be washed, dried and subjected to a grinding operation followed by calcination to produce the activated clay catalysts.

A substoichiometric amount of the heterogenous acid catalyst with respect to the starting material is typically used in the reaction. In some embodiments, the molar ratio of the heterogenous acid catalyst to levulinic acid is about 0.1:1. In some embodiments, the molar ratio of the heterogenous acid catalyst to levulinic acid is less than 0.1:1.

The methods of the invention provide angelica lactone in high yield. Typically, angelica lactone is obtained in yields ranging from at least 10% to greater than 95% yield. For example, angelica lactone can be obtained in yields of at least about 60%, or at least about 70%, or at least about 80%, or at least about 90%, or at least about 95%. In some embodiments, the angelica lactone is prepared in at least about 92% yield.

The angelica lactone can be prepared at any suitable pressure. In certain embodiments, the lactonization reaction is conducted at reduced pressure to facilitate distillation of the angelica lactone product. The angelica lactone can be prepared, for example, at pressures ranging between about 25 mmHg to about 250 mmHg. The angelica lactone can be prepared, for example, at from about 50 mmHg to about 225 mmHg, or from about 75 mmHg to about 200 mmHg, or from about 100 mmHg to about 175 mmHg, or from about 125 mmHg to about 150 mmHg, or from about 25 mmHg to about 150 mmHg, or from about 150 to about 250 mmHg. The angelica lactone can be prepared at about 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, or 250 mmHg. In some embodiments, the pressure of the reaction mixture is at most about 150 mmHg.

The angelica lactone can be prepared at any suitable temperature using the methods of the invention. In general, angelica lactone can be prepared at temperatures ranging between about 30° C. and about 250° C. The angelica lactone can be prepared, for example, at from about 50° C. to about 200° C., or from about 100° C. to about 175° C., or from about 40° C. to about 100° C., or from about 30° C. to about 150° C., or from about 100° C. to about 150° C., or from about 150° C. to about 200° C. The angelica lactone can be prepared at about 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245 or about 250° C. In some embodiments, the reaction mixture is heated to a temperature of from about 130° C. to about 200° C. In some embodiments, the reaction mixture is heated to at least about 165° C.

C. Methods for Preparing Angelica Lactone Dimer

In another aspect, the invention provides a method of preparing an angelica lactone dimer. The method includes forming a reaction mixture containing an angelica lactone and a catalytic amount of potassium carbonate under conditions sufficient to form the angelica lactone dimer, wherein the angelica lactone dimer is prepared in at least about 20% yield. The method is outlined in Scheme 3.

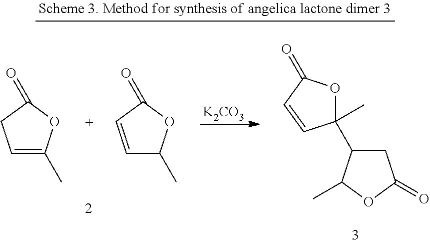

In some embodiments, the angelica lactone is selected from:

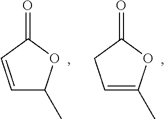

and mixtures thereof.

In some embodiments, the angelica lactone dimer has the formula:

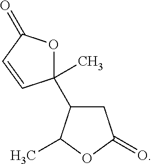

In some embodiments, the angelica lactone dimer consists essentially of

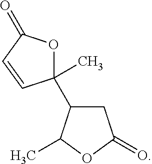

In some embodiments, the reaction mixture consists essentially of the angelica lactone and the potassium carbonate. Any suitable amount of potassium carbonate can be used in the methods of the invention. Typically, the reaction mixture for preparation of angelica lactone dimer contains from about 1 mol % to about 10 mol % potassium carbonate. The reaction mixture can contain, for example, from about 1 mol % to about 3 mol % potassium carbonate, or from about 3 mol % to about 5 mol % potassium carbonate, or from about 5 mol % to about 7 mol % potassium carbonate, or from about 7 mol % to about 9 mol % potassium carbonate, or from about 2 mol % to about 8 mol % potassium carbonate, or from about 4 mol % to about 6 mol % potassium carbonate. In some embodiments, the reaction mixture contains about 5 mol % potassium carbonate.

The methods of the invention provide angelica lactone dimer in high yield. In general, angelica lactone dimer is obtained it at least about 20% yield. angelica lactone dimer can be obtained, for example, in yields ranging from about 20% to about 95% or higher. The yield of angelica lactone dimer can be from about 20% to about 80%, or from about 40% to about 60%, or from about 55% to about 70%, or from about 70% to about 95%. In some embodiments, the angelica lactone dimer is prepared in at least about 50% yield. In some embodiments, the angelica lactone dimer is prepared in at least about 90% yield. In some embodiments, the angelica lactone dimer is prepared in about 94% yield.

The angelica lactone dimer in the methods of the invention can be prepared at any suitable temperature. In general, angelica lactone dimer can be prepared at temperatures ranging between about 30° C. and about 100° C. The angelica lactone dimer can be prepared, for example, at from about 30° C. to about 40° C., or from about 30° C. to about 70° C., or from about 40° C. to about 100° C., or from about 70° C. to about 100° C. The angelica lactone dimer can be prepared at about 30, 35, 40, 45, 50, 55, 60, 65, 70, 75, 80, 85, 90, 95, or about 100° C. In some embodiments, the reaction mixture is heated to a temperature of from about 50° C. to about 90° C. In some embodiments, the reaction mixture is heated to at least about 60° C. In some embodiments, the reaction mixture is heated to about 70° C.

D. Methods for Reducing Lactones

In another aspect, the invention provides a method of reducing a lactone to an alkane. The method includes forming a reaction mixture containing the lactone, a catalyst selected from Ir—ReOₓ/SiO₂ and Pt—ReOₓ/C, and hydrogen gas at a temperature of at least about 50° C. and a pressure greater than 1 bar, thereby reducing the lactone to the alkane. In some embodiments, the lactone includes an angelica lactone dimer. The alkane can be any alkane described herein, including C₆-C₁₀ alkanes and C₇-C₁₀ alkanes. In some embodiments, the alkane is a C₆-C₁₀ alkane or a mixture of C₆-C₁₀ alkanes.

In general, reduction reactions are conducted at pressures ranging between about 1 bar and about 75 bar. The reduction reaction can be conducted, for example, at from about 1 bar to about 10 bar, or from about 10 bar to about 25 bar, or from about 25 bar to about 50 bar, or from about 50 bar to about 75 bar, or from about 10 bar to about 60 bar, or from about 20 bar to about 50 bar, or from about 30 bar to about 40 bar. Typically, reduction reactions are conducted at temperatures ranging between about 50° C. and about 220° C. A reduction reaction can be conducted, for example, from about 50° C. to about 200° C., or from about 50° C. to about 150° C., or from about 100° C. to about 150° C., or from about 150° C. to about 220° C. A reduction reaction can be conducted at about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, or about 220° C. In some embodiments, the reduction reaction is conducted at a temperature of at least about 200° C.

Those of skill in the art will appreciate that any of the reactions described herein can be conducted with suitable cosolvents, including, but not limited to, diethyl ether, diisopropyl ether, ethyl acetate, pentane, hexane, heptane, cyclohexane, benzene, toluene, chloroform, dichloromethane, carbon tetrachloride, 1,2-dichloroethane, 1,1-dichloroethane, N,N-dimethylformamide, N,N-dimethylacetamide, dimethylsulfoxide, N-methyl 2-pyrrolidone, acetic acid, trifluoroacetic acid, trichloroacetic acid, methyl ethyl ketone, methyl isobutylketone, acetonitrile, propionitrile, 1,4-dioxane, sulfolane, 1,2-dimethyoxyethane, and combinations thereof. Any suitable reaction time can be used in the methods of the invention. In general, reactions are allowed to run for a time sufficient for consumption of the starting material and conversion to the desired product, or until conversion of the starting material comes to a stop. Depending on the configuration of the reactor, reactions are typically allowed to run for any amount of time ranging from a few minutes to several hours. Reactions can be run, for example, for anywhere between 2 minutes and 48 hours. Reactions can be run for about 20 minutes, or about 40 minutes, or about 60 minutes. Reactions can be run for about 1.5, 2, 2.5, 3, 3.5, 4, 4.5, 5, 5.5, or 6 hours. In some embodiments, reactions are run for less than 24 hours. In some embodiments, reactions are run for less than 12 hours. In some embodiments, reactions are run for less than 6 hours.

As described herein, the chemical and physical properties of the alkane products, such as high energy density, make them an attractive alternative to fossil fuels. Branched alkanes obtained by the methods of the invention can be blended with straight chain alkanes derived from simple sugars, as well as with aromatic compounds derived from renewable 2,5-dimethylfuran. Such mixtures can be used as fuels having the full component spectrum of conventional fuel (e.g., motor gasoline), while being obtained entirely from renewable resources.

### III. EXAMPLES

Materials and Methods.

Levulinic acid (>97%), montmorillonite K10, K₂CO₃ (>99%, anhyd.), copper(II) nitrate hemi(pentahydrate) (98%), zinc nitrate hexahydrate (98%), aluminum nitrate nonahydrate (>98%), 12-phosphotungstic acid (H₃O₄₀PW₁₂), ammonium rhenate (VII), and MCM-41 were purchased form Sigma Aldrich and used as received. Gamma-alumina (>97%) was purchased from Strem Chemicals. Silica gel (G-6, BET surface area 535 m²/g) was kindly supplied by Fuji Silysia Chemical Ltd. Dichloromethane, acetone, and HPLC grade water were purchased form Fischer Scientific and used without further purification. Calcination experiments were carried out using a Thermolyne type 2000 furnace. Catalytic hydrodeoxygenation experiments were performed in a 300 mL Parr pressure vessel (model 4766) fitted with a gas inlet with pressure gauge, thermocouple, safety valve and pressure release valve with a regulating stem for a controlled gas release.

### Example 1

### Preparation of Angelica Lactone from Levulinic Acid

Angelica lactone (AL) 2.

Levulinic acid 1 (20.01 g, 172.3 mmol) and montmorillonite K 10 (2.0 g, 10 wt %) were introduced into a round-bottomed flask with a magnetic stirrer bar. The flask was attached to a distillation setup with a 170 mm Vigreux column and the pressure in the system was reduced to 50 mm Hg using a vacuum pump with a pressure regulator. The mixture was heated in an oil bath with good stirring. The distillation started when the bath temperature reached 165° C. The clear liquid in the collecting flask was seen to separate into two phases. Dichloromethane (50 mL) was added and the organic layer was separated and dried over Na₂SO₄. Removal of the drying agent and evaporation of the solvent yielded a mixture of α (major) and β (minor) angelica lactones 2 as a clear liquid (15.60 g, 92%). The distillation flask was re-charged with fresh levulinic acid 1 and the reaction repeated twice in succession, giving AL 2 yields of 88 and 90%. ¹H NMR (CDCl₃, 300 MHz): 5.07 (d, J=1.5 Hz, 1H), 3.10 (t, J=2.7 Hz, 2H), 1.92 (d, J=1.5 Hz, 3H). ¹³C NMR (CDCl₃, 75 MHz): 177.1, 153.2, 99.4, 34.2, 14.1.

Levulinic acid (LA) 1 is one of the most recognizable products in the modern concept of the biorefinery. LA is on the US National Renewable Energy Laboratory top-twelve list of value added chemicals from biomass, as well as Bozell's new top-ten list of chemical opportunities from biorefinery carbohydrates. As shown in Scheme 4, LA 1 can undergo intramolecular dehydration to give a-angelica lactone (AL) 2, although the reaction has attracted relatively little interest in the renewables field, being largely eclipsed by the reduction and subsequent cyclization of 1 to γ-valerolactone [see, e.g., Wolff et al., Liebigs Ann. Chem. 1885, 229, 249; Alonso et al., Green Chem. 2013, 15, 584]. The reaction, as described, has generally involved the slow distillation of 2 from a mixture of 1 and a strong acid catalyst. The reaction gives AL in good yields but also results in a polymeric residue in the distillation pot, which presents problems for acid recycle on scale up. Using the methods of the present invention, a heterogeneous acid catalyst in this reaction facilitates product separation and catalyst recycling. This was accomplished using montmorillonite clay (K10), which gave >90% isolated yields of AL 2 without the formation of polymeric materials or noticeable deactivation of catalyst over three consecutive cycles. In a typical preparation, a mixture of LA and K10 (10 wt %) was distilled using a fractionating column under controlled vacuum (50 mmHg), resulting in a two-phase mixture of water and product, which could simply be separated.

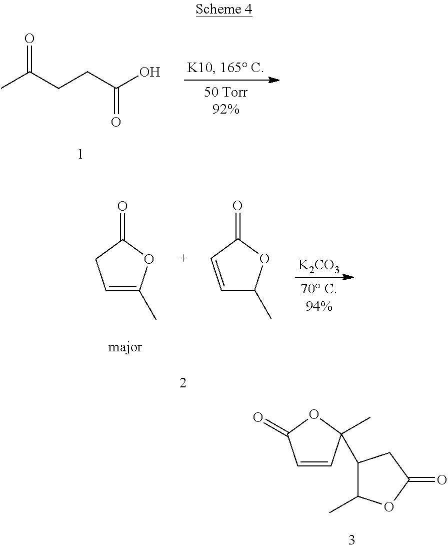

### Example 2

### Synthesis of Angelica Lactone Dimer

Angelica Lactone Dimer (ALD) 3.

Anhydrous K₂CO₃ (0.60 g, 4.3 mmol, 5 mol %) was added to angelica lactone 2 (10.00 g, 101.9 mmol) and the reaction flask was purged with argon and sealed. The suspension was placed in a pre-heated (70° C.) oil bath and stirred for 6 h. The mixture was cooled to room temperature and dichloromethane (100 mL) was added to the resulting thick paste with stirring. The K₂CO₃ was filtered off and the filtrate was washed with water (100 mL). The organic layer was separated, dried over Na₂SO₄ and the solvent was evaporated to give the angelica lactone dimer 3 as light yellow oil (9.3967 g, 94%). The liquid solidifies when refrigerated overnight to give a white crystalline solid. ¹H NMR (CDCl₃, 300 MHz): 7.36 (d, J=6.0 Hz, 1H), 6.16 (t, J=6.0 Hz, 1H), 4.45-4.32 (m, 1H), 2.73-2.44 (m, 2H), 2.34-2.28 (m, 1H), 1.51-1.37 (m, 6H). ¹³C NMR (CDCl₃, 75 MHz): 174.4, 171.3, 158.3, 157.9, 121.9, 121.7, 87.6, 87.2, 76.9, 76.3, 48.4, 47.8, 30.4, 30.1, 22.5, 22.4, 21.9, 21.4.

It is also known that AL 2 can dimerize, although again, the reaction is largely obscure. The angelica lactone dimer (ALD) 3 was first described in 1914 but not structurally characterized until 1954 [Losanitch et al., Compt. Rend. 1914, 158, 1683; Lukes et al., Coll. Czech. Chem. Commun. 1954, 19, 1205]. The dimerization reaction is a base-catalyzed conjugate addition between the double bond isomers of 2, which exist in equilibrium under the reaction conditions. The use of known catalysts—including hydroxide or alkoxide salts, active metals tertiary amines, and organometallics—were used in the present study with mixed results. A reaction employing anhydrous K₂CO₃ as the catalytic base was previously reported to yield the angelica lactone dimer 3 in 10.8% yield [Lukes et al., supra]. Surprisingly, the methods of the present invention provided angelica lactone dimer 3 in 94% yield.

### Example 3

### Synthesis of Alkanes from Angelica Lactone Dimer

Synthesis of Cu—ZnO/Al₂O₃ Catalyst.

The Cu—ZnO/Al₂O₃ catalyst was synthesized following a literature method [Peng et al., Chin. J. Catal. 2010, 31, 769] with minor modifications. Copper(II) nitrate hemipentahydrate (3.63 g), zinc nitrate hexahydrate (5.88 g), and aluminum nitrate nonahydrate (8.83 g) were dissolved in HPLC grade water (120 mL) to form solution A. In a separate flask, sodium carbonate (9.20 g) was dissolved in HPLC grade water (60 mL) to form solution B. Solution A was heated at 80° C. for 15 min, after which Solution B, which had been pre-heated to 80° C., was added dropwise over 45 min with rapid stirring. A thick, bluish-white precipitate appeared. After aging the mixture for 1 h to ensure complete co-precipitation, the solid was filtered under vacuum while hot and washed thoroughly using HPLC grade water (8×100 mL) until the pH of the washes was neutral. The precipitate was dried for 3 h in air at 100° C. to give a bluish-while solid. This solid was powdered using a mortar and pestle and then calcined at 500° C. for 3 h to give the final product as a black powder.

Synthesis of 12-phosphotungstic Acid (H₃O₄₀PW₁₂) Loaded on Mesoporous Silica (MCM-41).

The catalyst was prepared following a literature method [Varisli et al., Ind. Eng. Chem. Res. 2008, 47, 4071]. 12-Phosphotungstic acid (0.25 g) was dissolved in deionized water (12 mL) and MCM-41 (1.0 g) was added. The mixture was stirred at room temperature for 24 h. The water was evaporated under reduced pressure at 45° C. for 1 h and then at 60° C. for another hour. The resulting solid was dried at 160° C. for 4 h under reduced pressure (0.05 bar). Finally, the product was calcined at 300° C. for 3 h at a heating rate of 5° C./min.

Synthesis of the Ir—ReOₓ—SiO₂ Catalyst.

The synthesis of the bimetallic catalyst was adopted from a published procedure [Chen et al., ChemSusChem 2013, 6, 613]. An aqueous solution of IrCl₃ (135 mg, 0.45 mmol) in deionized water (5 mL) was added to silica gel (2.0 g, see Materials and Methods) and the mixture was stirred for 2 h at room temperature. The water was evaporated and the solid was dried at 110° C. for 24 h. The resulting material was stirred in a solution of NH₄ReO₄ (121 mg, 0.45 mmol) in water (5 mL) for 2 h. The water was evaporated under reduced pressure and the residue was dried overnight at 110° C. Calcination at 500° C. for 3 h at a heating rate of 2° C./min provided the catalyst.

Synthesis of the Pt—ReOₓ/C Catalyst.

An aqueous solution of NH₄ReO₄ (137 mg, 0.51 mmol) in water (5 mL) was added to commercial 5% Pt on activated carbon (2.0 g) and the mixture was stirred for 2 h at room temperature. The water was evaporated and the solid was dried at 110° C. for 24 h. The product was reduced under 7 bar H₂ pressure and 400° C. for 2 h. The catalyst was pre-reduced at 270° C. and 7 bar H₂ pressure for 1 h before use.

Catalytic hydrogenolysis of ALD 3 to hydrocarbons. angelica lactone dimer 3 (0.6-2.0 g), catalyst (10-15 wt %) and a magnetic stir bar were introduced into a 5 mL glass flask which was placed in a Parr pressure vessel. The reactor was sealed, flushed three times with hydrogen (30 bar) and finally pressurized to 54 bar. The pressure vessel was mounted in a heating mantle and heated to the reaction temperature, which was monitored using a thermocouple. Stirring was initiated when the pressure vessel reached the reaction temperature. After the reaction, heating and stirring were stopped and the vessel was allowed to cool to room temperature. The vessel was then further cooled to about −10° C. in an ice-salt bath and the pressure was slowly released through a bubbler filled with cold acetone. The vessel was opened and all parts were washed down with acetone. The two liquid samples were analyzed by GC-MS. Hydrocarbons in the bubbler represented between 0.7-1.1% of the total yield. Dodecane was used as an internal standard to quantify the products. The yield (in terms of C %) is as follows:

$${Yield}_{prd} ( \% ) = \frac{{mol}_{prd} \times C atoms in prd}{{mol}_{ALD 3} \times C atoms in ALD 3 ( = 10 )} \times 100$$

Product Analysis.

Liquid products were analyzed on a GC-MS (Agilent Technologies 6890N) equipped with a Varian Factor Four capillary column (VF-5ms, 30 m length, 0.25 mm inner diameter, 0.25 pm film). The injection temperature and the split ratio were 250° C. and 60:1, respectively. The oven temperature started at 50° C. and was held at that temperature for 2 min, then increased to 90° C. (2° C./min) and held at that temperature for 1 min, and finally increased to 300° C. (20° C./min) and held at that temperature for 2 min. The column pressure started at 31.2 kPa. The column flow was 1.0 mL/min. Commercial heptane (Sigma-Aldrich, 99%), octane (Sigma-Aldrich, ?99%), nonane (Sigma-Aldrich, >99%) and decane (Sigma-Aldrich, >99%) were used to calculate the response factors of C₇, C₈, C₉ and C₁₀ alkanes respectively against a dodecane standard. Mass spectrometry was performed using the electron impact ionization method, scanning from 40.0 to 350 m/z. The mass spectra of detected compounds were matched to MS database values from the National Institute of Standards and Technology (NIST).

Hexane, 3-methyl-(CAS# 589-34-4) MW 100, M.S (EI): m/z (% of max intensity) 41 (61) 43 (100), 55 (19), 56 (35), 57 (44), 70 (46), 71 (45). Retention time in GC/MS 2.62 min.

Heptane, 4-methyl-(CAS# 589-53-7) MW 114, M.S (EI): m/z (% of max intensity) 41 (27), 42 (13), 43 (100), 55 (15), 57 (14), 70 (46), 71 (53). Retention time in GC/MS 3.84 min.

Hexane, 3-ethyl-4-methyl-(CAS# 3074-77-9) MW 128, M.S (EI): m/z (% of max intensity) 41 (56), 43 (75), 55 (35), 57 (100), 70 (77), 71 (30). Retention time in GC/MS 6.08 min.

Heptane, 3,4-dimethyl-(CAS# 922-28-1) MW 128, M.S (EI): m/z (% of max intensity) 41 (48), 43 (100), 55 (22), 56 (42), 57 (66), 70 (47), 71 (30). Retention time in GC/MS 6.16 min.

Heptane, 3-ethyl, 4-methyl-(CAS# 52896-91-0) MW 142, M.S (ED: m/z (% of max intensity) 41 (37), 43 (93), 55 (36), 57 (63), 70 (100), 71 (79). Retention time in GC/MS 9.51 min.

The solvent and lighter alkanes were evaporated under reduced pressure to leave the product as a clear, pale yellow liquid. ¹³C NMR (CDCl₃, 75 MHz): 46.3, 36.6, 33.6, 23.3, 22.3, 21.1, 15.7, 14.7, 12.8, 12.6.

¹H NMR spectra were recorded using a Varian Merc 300 NMR spectrometer operating at 300 MHz. ¹³C NMR spectra were recorded on the same instrument with at an operating frequency of 75 MHz. The data were processed using MestReNova (version 6.2.0) desktop NMR data processing software.

Hydrocarbon Production Using Pt—ReO/C Bimetallic Catalyst.

The synthesis of Pt—ReOₓ/C bimetallic catalyst was adopted from a published procedure [Y. T. Kim, J. A. Dumesic, G. W. Huber, I Catal. 2013 304 (2013) 72-85]. The platinum on activated charcoal catalyst was purchased from Sigma Aldrich (5 wt % Pt/C). An aqueous solution of NH₄ReO₄ (137 mg, 0.51 mmol) in deionized water (5 mL) was added to 2 g of 5 wt % Pt/C and stirred for 2 h at room temperature. After evaporating the solvent and drying at 110° C. for 24 h, the resultant material was reduced at 400° C. for 2 h under a hydrogen flow.

Pt—ReOₓ/C catalyst (0.30 g, 15 wt %) and a magnetic stirring bar were introduced in a 5 mL glass flask, placed into the Parr pressure vessel and heated to 280° C. under H₂ (1 MPa) for 1 h for the reduction pretreatment. After the pretreatment, the autoclave was cooled and the H₂ was removed. ALD (2.00 g) was added to the 5 mL glass flask. The reactor was sealed, flushed three times with hydrogen (30 bar) and finally pressurized to 54 bar. The pressure vessel was placed in a heating mantle, and heated to the reaction temperature (240° C.). The temperature was controlled by using a thermocouple. Stirring was started when the pressure vessel reached the reaction temperature. The reaction was carried out at 240° C. for 7 h. After the reaction, heating and stirring were stopped and the vessel was allowed to come to room temperature. The vessel was further cooled down to about −10° C. using a salt-ice bath and the pressure release valve was connected to a bubbler filled with acetone. The hydrogen gas from the pressure vessel was bubbled slowly through the acetone trap. After the pressure inside attained atmospheric pressure, the vessel was opened and the parts were washed with acetone. The two acetone samples were analyzed by GC-MS. An internal standard (i.e. dodecane) was used to quantify the liquid products. The total yield of hydrocarbons (C₇-C₁₀) was 88% where the yield of 3-ethyl-4-methylheptane corresponds to 60% alone.

ALD 3 is a potentially valuable renewable feedstock for hydrodeoxygenation (HDO) due to its C₁₀ carbon count and branched character which, as described above, would make it an ideal precursor to cellulosic gasoline. Traditional hydrotreating catalysts such as sulfided NiMo and CoMo are commonly used for the reduction of fatty acid esters to hydrocarbons, but the gradual deactivation of these catalysts by sulfur leaching is disadvantageous [Senol et al., Catalysis Today 2005, 100, 331]. Copper-zinc mixed oxide catalyst on alumina (Cu-ZnO/Al₂O₃) has been used to hydrogenate esters to the corresponding alcohol in nearly quantitative yield under relatively mild conditions (230° C.) [He et al., Applied Catal. A: General 2013, 452, 88]. Since γ-Al₂O₃ is known to dehydrate alcohols under similar conditions [Nel et al., Ind. Eng. Chem. Res. 2007, 46, 3558], a combination of these two catalysts could achieve a one-pot conversion of ALD 3 to hydrocarbon. In a typical reaction, ALD 3 and the catalysts (10 wt % Cu—ZnO/Al₂O₃+20 wt % γ-Al₂O₃) were loaded into a pressure vessel and the system was pressurized with hydrogen to ca. 50 bar and heated with stirring at 300° C. for 3 h. Analysis of the reaction mixture showed a 41% yield of C₇ to C₁₀ hydrocarbons alongside a considerable quantity of alcohol and ether products. Since the dehydration of the intermediate alcohols was apparently not efficient enough, the γ-Al₂O₃ catalyst was replaced with phosphotungstic acid (H₃W₁₂O₄₀P) loaded on mesoporous silica (MCM-41), which is known to dehydrate aliphatic alcohols under even milder conditions than γ-Al₂O₃ [Herrera et al., Topics in Catalysis 2008, 49, 259]. When a combination of Cu—ZnO/Al₂O₃ and HPW/MCM-41 was employed, the total yield of hydrocarbons improved to 68%. Variation in catalyst loading, temperature, and reaction time did not improve the yield.

The hydrogenation of carbohydrate derivatives tends to favor noble metal catalysts, and recently it has been reported that such catalysts modified with an oxophilic metal like Re show higher catalytic activity in C—O bond hydrogenolysis [Chia et al., J. Am. Chem. Soc. 2011, 133, 12675]. In particular, an Ir—ReOₓ/SiO₂ catalyst described by Tomishige has demonstrated great promise in the reduction of glycitols to alkanes under mild conditions, along with good reusability. Remarkably, we have found that Ir—ReOₓ/SiO₂is also highly active in the HDO of lactones.

The Ir—ReOₓ/SiO₂ catalyst was prepared using the published method [Chen et al., ChemSusChem 2013, 6, 613] and reactions were carried out in batch mode as described above for the Cu—ZnO/Al₂O₃ system, except that a range of temperatures between 200 and 300° C. were studied. The results for the deoxygenation of ALD 3 are shown in Table 1. Starting at the same temperature as used for Cu—ZnO/Al₂O₃, an improved yield of 72% hydrocarbons was observed using the Ir—ReOₓ/SiO₂catalyst. A considerable degree of C-C bond cleavage was seen, as was the case with the copper-zinc catalyst, which prompted attempts to perform the hydrogenation at lower temperatures. As shown in Table 1, reduction of the reaction temperature first to 240 and then 220° C. increased selectivity for the C₁₀ product, i.e. the product which would result if no C—C bond cleavage occurred, while increasing the overall hydrocarbon yield up to 88%. Decreasing the reaction time (not shown) or the reaction temperature (200° C.) was found to substantially lower the yield of hydrocarbons.

TABLE 1
Angelica lactone dimer 3 conversion and hydrocarbon
yield with different catalytic systems.

| | Temp. | Time | Conv. | Hydrocarbon Yield (%)^{a} | | | | |
|---|---|---|---|---|---|---|---|---|
| Catalyst | (° C.) | (h) | (%) | C₇ | C₈ | C₉ | C₁₀ | Total |
| Cu—ZnO/Al₂O₃ + γ- | 300 | 3 | 100 | 3 | 12 | 5 | 21 | 41 |
| Al₂O₃ | | | | | | | | |
| Cu—ZnO/Al₂O₃ + | 300 | 3 | 100 | 4 | 15 | 35 | 14 | 68 |
| HPW/MCM-41^{b} | | | | | | | | |
| Ir—ReOₓ/SiO₂ | 300 | 3 | 100 | 4 | 16 | 19 | 33 | 72 |
| Ir—ReOₓ/SiO₂ | 240 | 6 | 100 | 1 | 13 | 10 | 60 | 84 |
| Ir—ReOₓ/SiO₂ | 220 | 7 | 100 | 1 | 11 | 6 | 70 | 88 |
| Ir—ReOₓ/SiO₂ | 200 | 7 | 100 | 0 | 6 | 1 | 3 | 10 |
| Pt—ReOₓ/C | 240 | 6 | 100 | 2 | 8 | 18 | 60 | 88 |
| Pt—ReOₓ/C | 220 | 7 | 100 | 2 | 4 | 7 | 13 | 26 |
| Pt—WOₓ/Al₂O₃ | 240 | 6 | 100 | 1 | 7 | 1 | 10 | 19 |

^{a}Molar % C yields as determined by GC-MS integration against m-alkane standards.
^{b}20 wt % 12-phosphotungstic acid (HPW) loaded on mesoporous silica (MCM-41).

The control of the hydrocarbon distribution with reaction temperature is rendered graphically in FIG. 2. Under the mild reaction conditions, a narrow range of products in the given carbon range resulted, as broken out in Scheme 5 for the reaction at 220° C. The carbon mass balance can be satisfied by taking decarbonylation (C₁₀→C₉ products) and/or ethyl group cleavage (C₁₀→C₈ and C₇ products) into account.

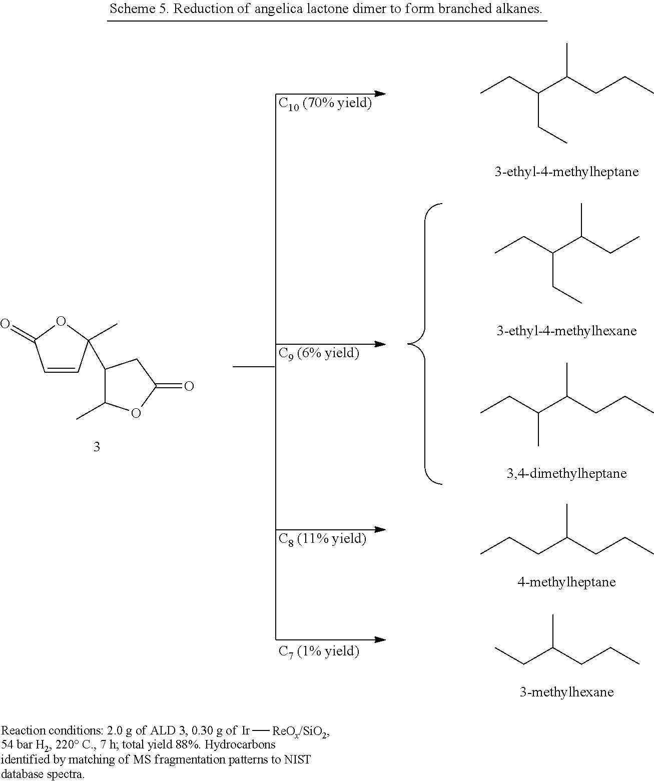

The robust nature of the Ir-based catalyst was also tested and its stability and reusability was studied. The results are shown in Table 2. Recovered catalyst was recycled three times to confirm its robust nature. It is important to note that the selectivities of the catalyst remained essentially unchanged.

TABLE 2
Reusability of Ir—ReOₓ/SiO₂ catalyst.

| | |
|---|---|
| Conv. | Hydrocarbon Yield (%)^{a} |

| Recycle | (%) | C₇ | C₈ | C₉ | C₁₀ | Total |
|---|---|---|---|---|---|---|
| 1 | 100 | 1 | 12 | 5 | 67 | 85 |
| 2 | 100 | 1 | 10 | 5 | 68 | 84 |
| 3 | 100 | 1 | 9 | 6 | 69 | 85 |

^{a}Reaction conditions: 2.0 g of ALD 3, 0.30 g of Ir—ReOₓ/SiO₂, 54 bar H₂, 220° C. and 7 h. Recovered catalyst was re-calcined between experiments.

The key considerations in the quest for economically competitive biofuel production center around issues of 1) yield, 2) feedstock, 3) process economics, and 4) market. Here, we introduce a process that addresses these matters as follows: 1) It operates in up to 76% overall yield in three steps from biomass-derived levulinic acid 1. Considering that 1 is available in >80% conversion from biomass, a field-to-tank yield of >60% is possible. 2) It does not involve impractical or difficult to access feedstocks (e.g. fructose, HMF). Economic projections have indicated that the production costs of LA 1 could be as low as $0.04-$0.10 lb, depending on the scale of the operation [Bozell et al., Resour. Conserv. Recycl. 2000, 28, 227]. 3) It proceeds under relatively mild conditions using cheap (K₁₀, K₂CO₃) and robust (Ir—ReOₓ/SiO₂) catalysts. 4) It represents the first synthesis of branched hydrocarbons in the gasoline volatility range from biomass. Central to market considerations is the concept of the “drop-in” product, which is only formally satisfied by a literal equivalent of the commercial product that seamlessly integrates into the prevailing transportation infrastructure. The C₇-C₁₀ iso-alkanes described here could be blended with C₄-C₆ n-alkanes that are available via other processes, such as the hydrogenation of simple sugars. The aromatic fraction of gasoline could be derived from renewable 2,5-dimethylfuran, which has a high RON (119) [Barlow et al., Eur. pat. EP0082689 1983], as well as being an oxygenate [S. Dutta, M. Mascal, ChemSusChem 2014, 7, 3028]. Thus, a basic combination of chemical-catalytic processes has the potential to supply essentially the full component spectrum of motor gasoline entirely from renewable resources.

The introduction of the angelica lactone dimer 3 as a new feedstock for HDO is made practical here by the development of a scalable approach to angelica lactone 2 itself and the production of 3 in nearly quantitative yield. Further, adaptation of the Ir—ReOₓ/SiO₂ catalyst to the HDO of cyclic esters, with branched alkane distribution governed by temperature, promises to encourage fresh efforts towards the identification of new renewable platform chemicals beyond sugars and furans.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A method for preparing a C₆-C₁₀ alkane, or a mixture thereof, comprising forming a reaction mixture comprising an angelica lactone dimer, a catalyst, and a hydrogen source under conditions sufficient to reduce the angelica lactone dimer, thereby preparing the alkane.

2. The method of claim 1, wherein the angelica lactone dimer is selected from the group consisting of: 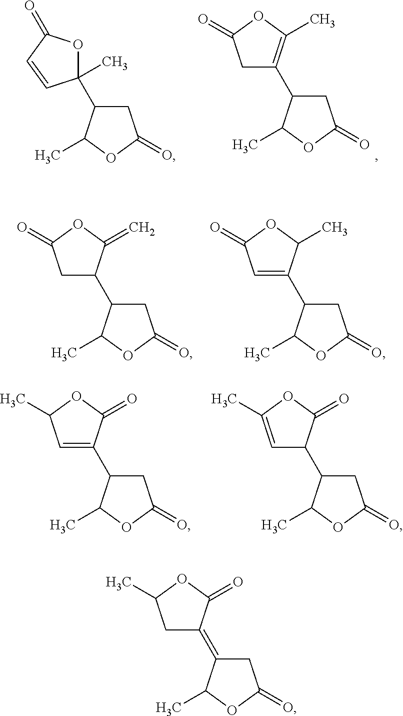
and mixtures thereof.

3. The method of claim 1, wherein the C₆-C₁₀ alkane includes an alkane selected from the group consisting of: 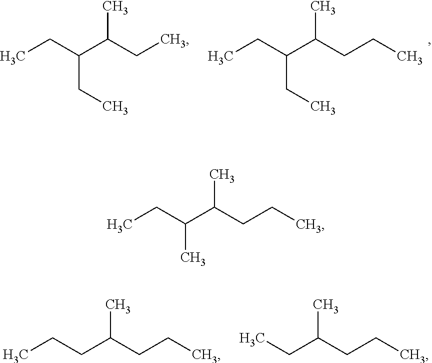
and mixtures thereof.

4. The method of claim 1, wherein the catalyst includes at least one member selected from the group consisting of CuO, ZnO, iridium metal, rhenium oxide (ReOₓ), platinum metal, tungsten oxide (WOₓ), and phosphotungstic acid.

5. The method of claim 1, wherein the reaction mixture is at a pressure of from about 1 bar to about 75 bar.

6. The method of claim 1, wherein the reaction mixture is heated to a temperature of from about 50° C. to about 300° C.

7. The method of claim 1, wherein the method further includes
forming a reaction mixture comprising an inorganic base and an angelica lactone selected from the group consisting of: 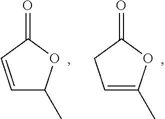
and mixtures thereof, under conditions sufficient to form the angelica lactone dimer.

8. The method of claim 7, wherein the inorganic base is K₂CO₃.

9. The method of claim 7, wherein the method further includes:
forming a reaction mixture comprising levulinic acid and a heterogeneous acid catalyst under conditions sufficient to form the angelica lactone.

10. The method of claim 9, wherein the heterogeneous acid catalyst includes montmorillonite clay.

11. The method of claim 9 comprising:
i) forming the reaction mixture comprising levulinic acid and montmorillonite clay under conditions sufficient to form the angelica lactone selected from the group consisting of: 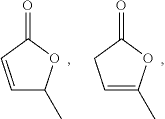
and mixtures thereof;
ii) forming the reaction mixture containing the angelica lactone and K₂CO₃ under conditions sufficient to form the angelica lactone dimer selected from the group consisting of 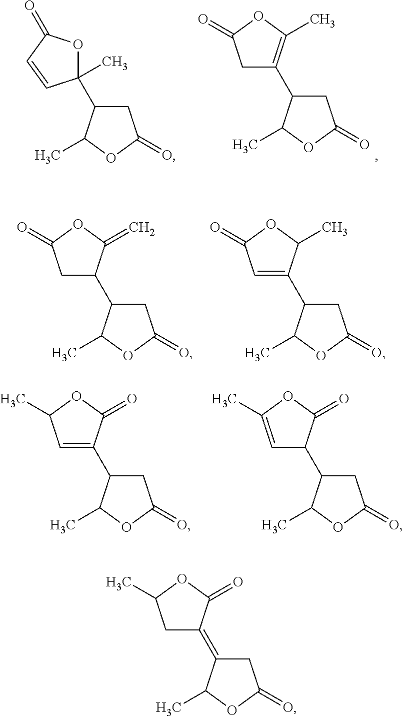
and mixtures thereof; and
iii) forming the reaction mixture containing the angelica lactone dimer, hydrogen gas, and a catalyst selected from the group consisting of Ir—ReOₓ/SiO₂ and Pt—ReOₓ/C, under conditions sufficient to form the C₆-C₁₀ alkane, wherein the C₆-C₁₀ alkane includes an alkane selected from the group consisting of: 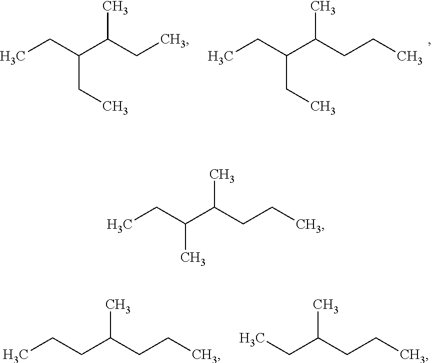
and mixtures thereof.

12. A C₆-C₁₀ alkane, or mixture thereof, prepared according to the method of claim 1.

13. A method of preparing an angelica lactone comprising forming a reaction mixture containing levulinic acid and a heterogeneous acid catalyst under conditions sufficient to lactonize the levulinic acid, thereby preparing the angelica lactone.

14. The method of claim 13, wherein the angelica lactone is selected from the group consisting of: 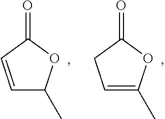
and mixtures thereof.

15. The method of claim 13, wherein the heterogeneous acid catalyst includes montmorillonite clay.

16. The method of claim 13, wherein the angelica lactone is prepared in at least about 90% yield.

17. A method of reducing a lactone to an alkane comprising forming a reaction mixture comprising the lactone, a catalyst selected from the group consisting of Ir—ReOₓ/SiO₂ and Pt—ReOₓ/C, and hydrogen gas at a temperature of at least about 50° C. and a pressure greater than 1 bar, thereby reducing the lactone to the alkane.

18. The method of claim 17, wherein the lactone includes an angelica lactone dimer.

19. The method of claim 17, wherein the temperature is at least about 200° C.

20. The method of claim 17, wherein the alkane is a C₆-C₁₀ alkane or a mixture thereof.
